(19) United States
(12) Patent Application Publication
(10) Pub. No.: US 2005/0003392 A1
(43) Pub. Date: Jan. 6, 2005

(54) Methods of small sample amplification

(75) Inventors: Susana Salceda, San Jose, CA (US); Kai Wu, Mountain View, CA (US); Natalia Briones, Sunnyvale, CA (US); Qing Bai, Santa Clara, CA (US); Yanxiang Cao, Mountain View, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/805,559

(22) Filed: Mar. 19, 2004

Related U.S. Application Data
(63) Continuation-in-part of application No. 10/763,414, filed on Jan. 26, 2004.
Application No. 10/763,414 is a continuation of application No. 09/738,892, filed on Dec. 18, 2000.
(63) Continuation-in-part of application No. 09/961,709, filed on Sep. 24, 2001, now Pat. No. 6,794,138.
Application No. 09/961,709 is a continuation-in-part of application No. 09/854,317, filed on May 11, 2001, now Pat. No. 6,582,938.
Application No. 09/961,709 is a continuation-in-part of application No. 09/738,892, filed on Dec. 18, 2000.
(60) Provisional application No. 60/172,340, filed on Dec. 16, 1999.

(51) Int. Cl. C12Q 1/68; C12P 19/34

(52) U.S. Cl. 435/6; 435/91.2

(57) ABSTRACT

The present invention relates to the amplification of nucleic acids, preferably from mRNA. A primer and promoter are added to a target sequence to be amplified and then the target is amplified in an in vitro transcription reaction and the product of this reaction is used as template for subsequent rounds of amplification. Polyadenylated control transcripts are added to the nucleic acid sample prior to the first step of amplification to monitor the efficiency of the amplification and labeling reactions.

### RELATED APPLICATIONS

The present application is a continuation-in-part of U.S. application Ser. No. 10/763,414 filed Jan. 26, 2004 and Ser. No. 09/961,709 filed Sep. 24, 2001 the disclosures of which are incorporated herein by reference.

### FIELD OF THE INVENTION

The present invention relates generally to the amplification of nucleic acids. More specifically, the present invention facilitates the amplification of mRNA for a variety of end uses.

### BACKGROUND OF THE INVENTION

Many biological functions are accomplished by altering the expression of various genes through transcriptional (e.g. through control of initiation, provision of RNA precursors, RNA processing, etc.) and/or translational control. For example, fundamental biological processes such as cell cycle progression, cell differentiation and cell death, are often characterized by the variations in the expression levels of a group of genes.

Gene expression is also associated with pathogenesis. For example, the lack of sufficient expression of functional tumor suppressor genes and/or the over expression of oncogene/protooncogenes could lead to tumorgenesis (Marshall, Cell, 64: 313-326 (1991); Weinberg, Science, 254: 1138-1146 (1991), incorporated herein by reference for all purposes). Thus, changes in the expression levels of particular genes (e.g. oncogenes or tumor suppressors) serve as signposts for the presence and progression of various diseases.

Highly parallel methods of monitoring the expression of a large number of genes in a biological sample are a valuable research and diagnostics tool. However, the amount of starting material that can be obtained from a given source is often limited and it is useful to amplify genetic material prior to analysis. Methods of amplification that allow analysis of a sample that may be too small for analysis without amplification facilitate the analysis of gene expression in small samples and possibly in a single cell.

### SUMMARY OF THE INVENTION

In one embodiment a method for measuring the relative abundance of a plurality of mRNAs in a nucleic acid sample is disclosed. The steps are as follows: (a) contact a nucleic acid sample comprising a plurality of different polyadenylated mRNAs with a first primer comprising poly d(T) and an RNA polymerase promoter; and, extend the first primer in a reaction mixture comprising reverse transcriptase to generate RNA:DNA duplexes; (b) synthesize second strand cDNA by incubating the RNA:DNA duplexes with a reaction mixture comprising DNA polymerase, RNase H and dNTPs, generating double stranded cDNA comprising an RNA polymerase promoter;(c) produce multiple copies of unlabeled antisense RNA by incubating the double stranded cDNA in a reaction mixture comprising an RNA polymerase, ATP, CTP, UTP and GTP; (d) purify the multiple copies of unlabeled antisense RNA; (e) contact the purified multiple copies of RNA with a reaction mixture comprising random primers; and, generating RNA:cDNA duplexes from the purified multiple copies of RNA by extending the random primers in a reaction mixture comprising a reverse transcriptase and dNTPs; (f) denature the RNA:cDNA duplexes; (g) contact the DNA with a second primer comprising oligo dT and an RNA polymerase promoter and extending the second primer to generate double stranded cDNA; (h) form a double stranded DNA promoter region by adding the appropriate reagents; (i) produce multiple copies of labeled antisense cRNA by an in vitro transcription reaction; (j) fragment the labeled antisense cRNA; (k) hybridize the fragmented labeled antisense cRNA to a solid support comprising nucleic acid probes, wherein the probes are subsequences of a plurality of mRNAs; and (1) analyze the hybridization pattern to provide a measurement of the relative abundance of a plurality of mRNAs in the nucleic acid sample.

In another embodiment a known amount of at least one polyadenylated control transcript is added to the nucleic acid sample prior to step (a). The polyadenylated control transcript is preferably one not naturally present in the nucleic acid sample. The control transcript is taken through each of the steps of amplification and labeling and can be used to determine how efficiently the amplification and labeling steps have been performed. There are many opportunities to introduce variability into the assay, for example, the amplification may be inefficient, cleanup steps may result in loss of signal and labeling may also result in reduction of signal. Including controls that are present at known amounts allows for identification of problems and troubleshooting.

In many embodiments the control transcripts are from prokaryotic organisms where the nucleic acid sample is from a eukaryotic organism. Controls from a eukaryotic organism may be used when a prokaryotic organism is analyzed. The prokaryotic organism may be, for example, B. subtilis. In preferred embodiments two or more control transcripts are included and they are added to the nucleic acid sample at different concentrations.

In one embodiment the polyadenylated control transcripts are from a gene selected from the group consisting of B. subtilis lys, phe, thr and dap and the solid support comprises probes to detect at least one transcript from B. subtilis lys, phe, thr and dap. In a preferred embodiment transcripts from each of these genes are used and probes to detect each of the genes are included on the solid support.

In preferred embodiments the solid support may be a nucleic acid probe array, a membrane blot, a microwell, a bead, or a sample tube.

In preferred embodiments the nucleic acid sample is obtained from tissue, blood or a buccal swab. Blood samples and buccal swab samples are easy to obtain and are less invasive to ways of obtaining a sample from an individual.

In some embodiments the steps of the methods involve the use of a thermocycler, an integrated reaction device, and a robotic delivery system. In preferred embodiments the samples may be processed by high throughput methods. In one embodiment kits for the amplification of nucleic acids are disclosed. The kits may contain a container, instructions for use, a promoter which comprises a poly d(T) sequence operably linked to an RNA polymerase promoter and at least one polyadenylated control transcript from a gene from a prokaryotic organism. A preferred kit contains polyadenylated control transcripts from the B. subtilis lys, phe, thr and dap genes.

In one embodiment a method for measuring the relative abundance of a plurality of mRNAs in a nucleic acid sample is disclosed. The steps of the method are: (a) obtaining a nucleic acid sample wherein the nucleic acid sample comprises a mixture of polyadenylated mRNAs wherein at least one of the mRNAs is present in the nucleic acid sample at unknown levels; (b) adding a known amount of at least one polyadenylated control transcript to the nucleic acid sample, wherein the at least one polyadenylated control transcript is not naturally present in the nucleic acid sample, to form a mixed nucleic acid sample; (c) contacting the mixed nucleic acid sample with a first primer comprising poly d(T) and an RNA polymerase promoter; and, extending the first primer in a reaction mixture comprising reverse transcriptase to generate RNA:cDNA duplexes; (d) synthesizing second strand cDNA by incubating the RNA:cDNA duplexes with a reaction mixture comprising DNA polymerase, RNase H and dNTPs, generating double stranded cDNA comprising an RNA polymerase promoter; (e) producing multiple copies of unlabeled antisense RNA by incubating the double stranded cDNA in a reaction mixture comprising an RNA polymerase, ATP, CTP, UTP and GTP; (f) purifying the multiple copies of unlabeled antisense RNA; (g) contacting the purified multiple copies of RNA with a reaction mixture comprising random primers; and, generating RNA:cDNA duplexes from the purified multiple copies of unlabeled antisense RNA by extending the random primers in a reaction mixture comprising a reverse transcriptase and dNTPs; (h) denaturing the RNA:cDNA duplexes; (i) contacting the cDNA with a second primer comprising oligo dT and an RNA polymerase promoter and extending the second primer to generate double stranded cDNA; (j) forming a double stranded DNA promoter region by adding the appropriate reagents; (k) producing multiple copies of labeled antisense cRNA by an in vitro transcription reaction; (l) fragmenting the labeled antisense cRNA; (m) hybridizing the fragmented labeled antisense cRNA to a solid support comprising nucleic acid probes, wherein the probes comprise probes that are subsequences of a plurality of mRNAs and probes that are subsequences of the at least one polyadenylated control transcript added in step (b) and (n) analyzing the hybridization pattern of the probes that are subsequences of a plurality of mRNAs to provide a measurement of the relative abundance of a plurality of mRNAs in the nucleic acid sample and analyzing the hybridization pattern of the probes that are subsequences of the at least one control transcript to measure the efficiency of one or more steps selected from steps (c) through (k).

### BRIEF DESCRIPTION OF THE FIGURES

FIG. 1 shows a schematic of a two-cycle target labeling assay.

FIG. 2 shows a schematic of a one-cycle target labeling assay.

### DETAILED DESCRIPTION OF THE PREFERRED EMBODIMENTS

General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT applications Nos. PCT/US99/00730 (International Publication Number WO 99/36760) and PCT/US01/04285, which are all incorporated herein by reference in their entirety for all purposes. See also, Fodor et al., Science 251(4995), 767-73, 1991, Fodor et al., Nature 364(6437), 555-6, 1993 and Pease et al. PNAS USA 91(11), 5022-6, 1994 for methods of synthesizing and using microarrays.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods are shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. patent application Publication No. 20030036069), and U.S. Pat. Nos. 5,856,092, 6,300,063, 5,858,659, 6,284,460, 6,361,947, 6,368,799 and 6,333,179. Additional methods of genotyping, complexity reduction and nucleic acid amplification are disclosed in U.S. patent application Ser. Nos. 60/508,418, 60/468,925, 60/493,085, 09/920,491, 10/442,021, 10/654,281, 10/316,811, 10/646,674, 10/272,155, 10/681,773 and 10/712,616 and U.S. Pat. No. 6,582,938. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and W088/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. patent application Publication No. 20030096235), Ser. No. 09/910,292 (U.S. patent application Publication No. 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. No. 60/364,731 and in PCT application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (U.S. Publication No. 20020183936), Ser. Nos. 10/065,856, 10/065,868, 10/328,818, 10/328,872, 10/423,403, and 60/482,389.

Additionally, gene expression monitoring and sample preparation methods can be shown in U.S. Pat. Nos. 5,800,992, 6,040,138, and 6,013,449.

Definitions

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization conditions will typically include salt concentrations of less than about 1 M, more usually less than about 500 mM and preferably less than about 200 mM. For example, conditions of 5×SSPE (750 mM NaCl, 50 mM NaPhosphate, 5 mM EDTA, pH 7.4) and a temperature of 25-30° C. are suitable for allele-specific probe hybridizations. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Hybridization conditions for microarrays are also disclosed in GeneChip Expression Analysis Technical Manual available from Affymetrix (April, 2003) Part No. 701045 Rev. 3.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Oligonucleotides may be chemically synthesized and may include modifications. Amino modifier reagents may be used to introduce a primary amino group into the oligo. A primary amino group is useful for a variety of coupling reactions that can be used to attach various labels to the oligo. The most frequently used labels are in the form of NHS-esters, which can couple with primary amino groups. A variety of derivatives of biotin are available in which the biotin moiety is connected (through the 4-carboxybutyl group) to a linker molecule that can be attached directly to an oligonucleotide. Fluorescent dies such as 6-FAM, HEX, TET, TAMRA, and ROX may be coupled to an oligo. Phosphate groups may be attached to the 5′ and/or 3′ end of an oligo. Oligos may also be phosphorothioated. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified “backbone” of an S-Oligo is resistant to the action of most exonucleases and endonucleases. In some embodiments the oligo is sulfurized only at the last few residues at each end of the oligo. This results in an oligo that is resistant to exonucleases, but has a natural DNA center. Degenerate bases may also be incorporated into an oligo. may also be incorporated into an oligo Additional modifications that are available include, for example, 2′ O-Methyl RNA, 3′-Glyceryl, 3′-Terminators, Acrydite, Cholesterol labeling, Dabcyl, Digoxigenin labeling, Methylated nucleosides, Spacer Reagents, Thiol Modifications DeoxyInosine, DeoxyUridine and halogenated nucleosides.

Probe: A probe is a surface-immobilized molecule that can be recognized by a particular target. Examples of probes include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies. Preferred probes are nucleic acids, preferably DNA, that are complementary to the antisense RNA that is produced by the methods of the invention. In preferred embodiments the probes are subsequences of the mRNAs to be detected, meaning that the probes are short sequences, between 15 and 100 bases, preferably 25, and the sequence is present in the mRNA to be detected and therefore the probes are complementary to a region of the antisense cRNA. Arrays comprising all possible probes sequences of a given length are disclosed in U.S. Pat. No. 6,582,908.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The Process

In general, the presently preferred invention enables a user to amplify mRNA (a target sequence) for gene expression monitoring experiments. Although one of skill in the art will recognize that other uses may be made of the amplified nucleic acid. In one embodiment of the current invention mRNA is contacted with a poly d(T) primer preferably having an RNA polymerase promoter attached to the poly d(T).

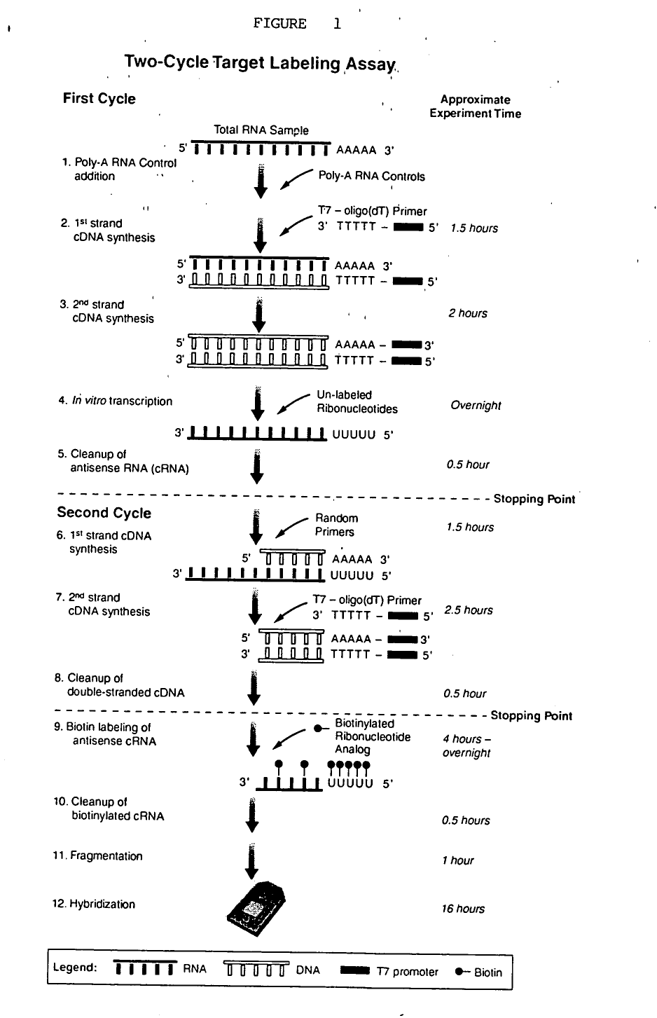

A preferred embodiment shown in FIG. 1 has the following steps: step 1: poly-A RNA control addition, step 2: 1^{st} strand cDNA synthesis, step 3: 2^{nd} strand cDNA synthesis, step 4. in vitro transcription, step 5: cleanup of antisense RNA (cRNA), step 6. 1^{st} strand cDNA synthesis, step 7: 2^{nd} strand cDNA synthesis, step 8: cleanup of double-stranded cDNA, step 9: biotin labeling of antisense cRNA, step 10: cleanup of biotinylated cRNA, step 11: fragmentation of cRNA, and step 12 hybridization to an array. The first cycle comprises steps 1 to 5 and the second cycle comprises steps 6 to 8. In some embodiments, step 8, the cleanup of double-stranded cDNA is left out and the assay goes directly from step 7 to step 9, in a preferred embodiment the reaction is heated between steps 7 and 9, for example, at 75° C. for 10 minutes then cooled to 4° C. for about at least 2 min.

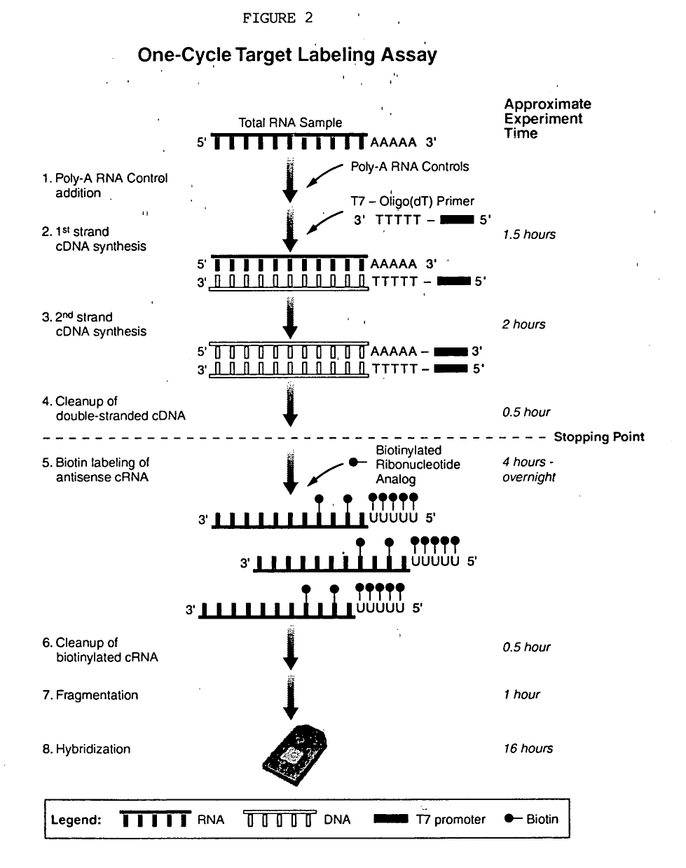

Another embodiment shown in FIG. 2 has the following steps: step 1 poly-A RNA control addition, step 1, 1^{st} strand cDNA synthesis, step 3, 2^{nd} strand cDNA synthesis, step 4, cleanup of double-stranded cDNA, step 5, biotin labeling of antisense cRNA, step 6, cleanup of biotinylated cRNA, step 7, fragmentation, step 8 hybridization to an array. In one embodiment step 4, cleanup of double stranded cDNA is omitted and the method goes directly from step 3 to step 5 without a cleanup step, in the place of the cleanup step a heat inactivation step may be incorporated, for example, 75° C. for 10 minutes then cooled to 4° C. for about at least 2 min.

In one embodiment mRNA is contacted with a poly d(T) primer attached to a promoter sequence. A first DNA strand is synthesized using an RNA dependent DNA polymerase and a second DNA strand is synthesized using DNA polymerase, forming an operable promoter. Thereafter, the appropriate reagents are added to transcribe the target portion in an IVT reaction to synthesize antisense RNA. In some embodiments steps are taken to clean the antisense RNA prior to subsequent manipulation, for example, prior to use in a second round of amplification. Cleanup may be, for example, column chromatography, phenol extraction, and ethanol precipitation. One of skill in the art will be familiar with methods of cleanup of nucleic acids. The cleanup procedure will typically remove proteins and small molecules that, for example, may inhibit downstream reactions, although some residual contaminants may remain after cleanup.

In many embodiments the RNA generated from the first IVT reaction is used as template for a second round of amplification. In the second round of amplification random primers are added and a second, single stranded DNA is synthesized with reverse transcriptase using the antisense RNA as a template. The RNA-DNA duplex is denatured and the DNA is contacted with an oligonucleotide sequence, which comprises poly d(T) and a functional promoter. The oligonucleotide is extended to make a second strand DNA and the first strand DNA is filled in (made double stranded) so that there is a functional promoter operably linked to the target sequence. Thereafter, the appropriate reagents are added to transcribe the target portion in an IVT reaction. Alternatively, the oligonucleotide sequence may be constructed so that it does not serve as a primer for extension of a sequence that is complementary to the target sequence, i.e. it is blocked.

In one embodiment, the invention is as follows: PolyA+ containing mRNA or total RNA is mixed with polyadenylated control transcripts in known amounts and the mixture is annealed with the single-stranded oligo d(T)-tailed primer with a promoter sequence, such as TₓNₓ, where Nₓ comprises an RNA polymerase promoter sequence such as the T7 promoter sequence, creating a primer-template mixture. T3 and SP6 promoter sequences may also be used. First strand cDNA synthesis may be accomplished by combining the first strand cDNA reagent mix (Superscript II buffer, DTT, and dNTPs) and enzyme mix (SuperScript II, ThernoScriptase, and RNAout) with the primer-template mixture and incubating at the appropriate time and temperature. A second strand cDNA is then formed by mixing the first strand cDNA reaction with random primers and second strand reagent mix, containing secondary cDNA mix (DEPC-H₂0, Tris-HCl (pH7.0), MgCl₂, (NH₄)SO₄, beta-NAD⁺, and dNTPs) and cDNA enzyme mix (Vent DNA polymerase, Amplitaq DNA polymerase, E. coli ligase, E. coli RNase H, and E. coli DNA polymerase I), followed by incubation at the appropriate times and temperatures. The resulting double-stranded (ds) cDNA contains a functional T7 RNA polymerase promoter, which is utilized for transcription. In vitro transcription (IVT) is performed by combining the ds cDNA with IVT reagent (buffer, NTP, DTT, RNase inhibitor, and T7 RNA polymerase), yielding amplified, antisense RNA, which is preferably unlabeled.

The RNA product of the first round of amplification is then used as template for a second round of amplification. Random primers are hybridized to the RNA creating a primer-template mixture. First strand cDNA synthesis is accomplished by combining the first strand cDNA reagent mix (Superscript II buffer, DTT, and dNTPs) and enzyme mix (SuperScript, ThermoScriptase, and RNAout) with the primer-template mixture and incubating at the appropriate time and temperature. The resultant RNA:cDNA duplex is then denatured and mixed with an oligo d(T)-tailed primer with a promoter sequence, such as TₓNₓ, where Nₓ comprises an RNA polymerase promoter sequence. Formation of a DNA strand that can serve as a template for an IVT reaction is then accomplished by combining the promoter primer-template mixture with Klenow fragment of E. coli DNA polymerase I and T4 DNA polymerase and incubating at the appropriate times and temperatures (only the promoter region needs to be double stranded). The resulting ds cDNA contains a functional T7 RNA polymerase promoter, which is utilized for transcription. In vitro transcription (IVT) is performed by combining the ds cDNA with IVT reagent (buffer, NTP, DTT, RNase inhibitor, and T7 RNA polymerase), yielding amplified, antisense RNA. The second round of amplification may be repeated one or more times. (See also U.S. Pat. No. 6,582,906.)

The present invention can be combined with other processes to eliminate the need for multiple steps and varying reaction conditions and their associated problems. (See, e.g., PCT/US00/20563, which is hereby incorporated by reference in its entirety.) In preferred embodiments of the present invention, at least three otherwise separate enzymatic reactions can occur consecutively in one phase (i.e., without organic extraction and precipitation), more preferably in the same reaction vessel. Preferably, cDNA synthesis according to the present invention may occur in a modified low salt buffer. In addition, the invention may involve an enzyme mix, which may include a thermal stable DNA polymerase and reverse transcriptase for the production of cDNA, and RNA polymerase for RNA transcription. Enzyme activity may be inactivated at the appropriate step with either heat or chemical treatment (for example, adjusting the salt concentration) or by the addition of an antibody specific to the enzyme.

Those skilled in the art will recognize that the products and methods embodied in the present invention may be applied to a variety of systems, including commercially available gene expression monitoring systems involving nucleic acid probe arrays, membrane blots, microwells, beads, and sample tubes, constructed with various materials using various methods known in the art. Accordingly, the present invention is not limited to any particular environment, and the following description of specific embodiments of the present invention are for illustrative purposes only.

The reaction vessel according to the present invention may include a membrane, filter, microscope slide, microwell, sample tube, array, or the like. (See International Patent applications Nos. PCT/US95/07377 and PCT/US96/11147, which are expressly incorporated herein by reference.) The reaction vessel may be made of various materials, including polystyrene, polycarbonate, plastics, glass, ceramic, stainless steel, or the like. The reaction vessel may preferably have a rigid or semi-rigid surface, and may preferably be conical (e.g., sample tube) or substantially planar (e.g., flat surface) with appropriate wells, raised regions, etched trenches, or the like. The reaction vessel may also include a gel or matrix in which nucleic acids may be embedded. (See A. Mirzabekov et al., Anal. Biochem. 259(1):34-41 (1998), which is expressly incorporated herein by reference.)

The nucleic acid sample according to the present invention may refer to any mixture of two or more distinct species of single-stranded mRNA, DNA or double-stranded DNA, which may include DNA representing genomic DNA, genes, gene fragments, oligonucleotides, polynucleotides, nucleic acids, PCR products, expressed sequence tags (ESTs), or nucleotide sequences corresponding to known or suspected single nucleotide polymorphisms (SNPs), having nucleotide sequences that may overlap in part or not at all when compared to one another. The species may be distinct based on any chemical or biological differences, including differences in base composition, order, length, or conformation. The single-stranded DNA population may be isolated or produced according to methods known in the art, and may include single-stranded cDNA produced from a mRNA template, single-stranded DNA isolated from double-stranded DNA, or single-stranded DNA synthesized as an oligonucleotide. The double-stranded DNA population may also be isolated according to methods known in the art, such as PCR, reverse transcription, and the like.

Where the nucleic acid sample contains RNA, the RNA may be total RNA, poly(A)⁺ RNA, mRNA, rRNA, or tRNA, and may be isolated according to methods known in the art. (See, e.g., T. Maniatis et al., Molecular Cloning: A Laboratory Manual, 188-209 (Cold Spring Harbor Lab., Cold Spring Harbor, N.Y. 1982, which is expressly incorporated herein by reference.) The RNA may be heterogeneous, referring to any mixture of two or more distinct species of RNA. The species may be distinct based on any chemical or biological differences, including differences in base composition, length, or conformation. The RNA may contain full length mRNAs or mRNA fragments (i.e., less than full length) resulting from in vivo, in situ, or in vitro transcriptional events involving corresponding genes, gene fragments, or other DNA templates. In a preferred embodiment, the mRNA population of the present invention may contain single-stranded poly(A)+ RNA, which may be obtained from an RNA mixture (e.g., a whole cell RNA preparation), for example, by affinity chromatography purification through an oligo-dT cellulose column.

Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993), all of which are incorporated herein by reference in their entireties for all purposes.

In a preferred embodiment, the total RNA is isolated from a given sample using, for example, an acid guanidinium-phenol-chloroform extraction method and polyA⁺ mRNA is isolated by oligo dT column chromatography or by using (dT)n magnetic beads. (See e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). (See also PCT/US99/25200 for complexity management and other sample preparation techniques, which is hereby incorporated by reference in its entirety for all purposes.) Where the single-stranded DNA population of the present invention is cDNA produced from a mRNA population, it may be produced according to methods known in the art. (See, e.g, Maniatis et al., supra, at 213-46.) In a preferred embodiment, a sample population of single-stranded poly(A)+ RNA may be used to produce corresponding cDNA in the presence of reverse transcriptase, oligo-dT primer(s) and dNTPs. Reverse transcriptase may be any enzyme that is capable of synthesizing a corresponding cDNA from an RNA template in the presence of the appropriate primers and nucleoside triphosphates. In a preferred embodiment, the reverse transcriptase may be from avian myeloblastosis virus (AMV), Moloney murine leukemia virus (MMuLV) or Rous Sarcoma Virus (RSV), for example, and may be thermal stable enzyme (e.g., rTth DNA polymerase available from PE Applied Biosystems, Foster City, Calif.).

Reverse transcriptase (e.g., either derived from AMV or MuLV) is available from a large number of commercial sources including Invitrogen/LTI, Amersham Phamacia Biotech (APB)/USB, Qiagen, and others. Other enzymes required or desired are also available from these vendors among others, such as Promega, and Epicentre. Nucleotides such as dNTPs, unique nucleotide sequences, and β-NAD are available from a variety of commercial sources such as APB, Roche Biochemicals, Sigma Chemicals. Buffers, salts and cofactors required or desired for these reactions can usually be purchased from the vendor that supplies a respective enzyme or assembled from materials commonly available, e.g., from Sigma Chemical.

In a preferred embodiment of the present invention, the ends of the double-stranded DNA may be blunted. T4 DNA polymerase or E. coli DNA polymerase I (Klenow fragment), for example, may be used preferably to produce blunt ends in the presence of the appropriate dNTPs.

Multiple copies of RNA according to the present invention may be obtained by in vitro transcription from the DNA preferably using T7 RNA polymerase in the presence of the appropriate nucleoside triphosphates. In a preferred embodiment of the present invention, the multiple copies of RNA may be labeled by the incorporation of biotinylated, fluorescently labeled or radiolabeled CTP or UTP during the RNA synthesis. (See U.S. Pat. Nos. 5,800,992, 6,040,138 and International patent application PCT/US96/14839, which is expressly incorporated herein by reference. Alternatively, labeling of the multiple copies of RNA may occur following the RNA synthesis via the attachment of a detectable label in the presence of terminal transferase. In a preferred embodiment of the present invention, the detectable label may be radioactive, fluorometric, chemiluminescent, enzymatic, or colorimetric, or a substrate for detection (e.g., biotin). Other detection methods, involving characteristics such as scattering, IR, polarization, mass, and charge changes, may also be within the scope of the present invention.

In a preferred embodiment, the amplified DNA or RNA of the present invention may be analyzed with a gene expression monitoring system. Several such systems are known. (See, e.g., U.S. Pat. No. 5,677,195; Wodicka et al., Nature Biotechnology 15:1359-1367 (1997); Lockhart et al., Nature Biotechnology 14:1675-1680 (1996), which are expressly incorporated herein by reference.) A preferred gene expression monitoring system according to the present invention may be a nucleic acid probe array, such as the GeneChip® nucleic acid probe array (Affymetrix, Santa Clara, Calif.). (See, U.S. Pat. Nos. 5,744,305, 5,445,934, 5,800,992, 6,040,193 and International patent applications PCT/US95/07377, PCT/US96/14839, and PCT/US96/14839, which are expressly incorporated herein by reference. A nucleic acid probe array preferably comprises nucleic acids bound to a substrate in known locations. In other embodiments, the system may include a solid support or substrate, such as a membrane, filter, microscope slide, microwell, sample tube, bead, bead array, or the like. The solid support may be made of various materials, including paper, cellulose, gel, nylon, polystyrene, polycarbonate, plastics, glass, ceramic, stainless steel, or the like including any other support cited in U.S. Pat. Nos. 5,744,305 or 6,040,193. The solid support may preferably have a rigid or semi-rigid surface, and may preferably be spherical (e.g., bead) or substantially planar (e.g., flat surface) with appropriate wells, raised regions, etched trenches, or the like. The solid support may also include a gel or matrix in which nucleic acids may be embedded. The gene expression monitoring system, in a preferred embodiment, may comprise a nucleic acid probe array (including an oligonucleotide array, a cDNA array, a spotted array, and the like), membrane blot (such as used in hybridization analysis such as Northern, Southern, dot, and the like), or microwells, sample tubes, beads or fibers (or any solid support comprising bound nucleic acids). See U.S. Pat. Nos. 5,770,722, 5,744,305, 5,677,195 5,445,934, and 6,040,193 which are incorporated here in their entirety by reference. (See also Examples, infra.) The gene expression monitoring system may also comprise nucleic acid probes in solution.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue. (See U.S. Pat. Nos. 5,800,922 and 6,040,138.) In a preferred embodiment, the proportional amplification methods of the present invention can provide reproducible results (i.e., within statistically significant margins of error or degrees of confidence) sufficient to facilitate the measurement of quantitative as well as qualitative differences in the tested samples. The proportional amplification methods of the present invention may also facilitate the identification of single nucleotide polymorphisms (SNPs) (i.e., point mutations that can serve, for example, as markers in the study of genetically inherited diseases) and other genotyping methods from limited sources. (See e.g., Collins et al., 282 Science 682 (1998), which is expressly incorporated herein by reference.) The mapping of SNPs can occur by any of various methods known in the art, one such method being described in U.S. Pat. No. 5,679,524, which is hereby incorporated by reference.

The RNA, single-stranded DNA, or double-stranded DNA population of the present invention may be obtained or derived from any tissue or cell source. Indeed, the nucleic acid sought to be amplified may be obtained from any biological or environmental source, including plant, virion, bacteria, fungi, or algae, from any sample, including body fluid or soil. In one embodiment, eukaryotic tissue is preferred, and in another, mammalian tissue is preferred, and in yet another, human tissue is preferred. The tissue or cell source may include a tissue biopsy sample, a cell sorted population, cell culture, or a single cell. In a preferred embodiment, the tissue source may include brain, liver, heart, kidney, lung, spleen, retina, bone, lymph node, endocrine gland, reproductive organ, blood, nerve, vascular tissue, and olfactory epithelium. In yet another preferred embodiment, the tissue or cell source may be embryonic or tumorigenic. In preferred embodiments the nucleic acid sample may be isolated from a blood sample or a buccal swab. When isolating nucleic acids from blood it may be advantageous to take steps to reduce the amplification of nucleic acids that may interfere with downstream analysis, for example, rRNA, tRNA or globin mRNAs. Certain RNAs that are present at high levels may interfere with the analysis of mRNAs that are present at lower levels and the high abundance messages may be preferentially degraded or their amplification may be preferentially inhibited. See, for example, US Pat. Nos. 6,391,592 and 6,410,229 and U.S. patent application Ser. No. 10/684,205.

Methods of isolating RNA are well known in the art. In one embodiment total RNA is isolated from yeast using a hot phenol protocol as described in Schmitt, et al. Nucl Acids Res 18:3091-3092 (1990). For isolation of total RNA from Arabidopsis TRIzol Reagent from Invitrogen Life Technologies may be used. In some embodiments RNA may be isolated from mammalian cells (such as cultured cells and lymphocytes) using the RNeasy Mini Kit from QIAGEN. When mammalian tissue is used as a source, in some embodiments, total RNA is isolated with a commercial reagent, such as TRIzol. A second cleanup step may be employed if going directly from TRIzol-isolated RNA to cDNA synthesis. In many embodiments, mRNA may be isolated using QIAGEN's oligotex mRNA Kit.

The materials for use in the present invention are ideally suited for the preparation of a kit suitable for the amplification of nucleic acids. Such a kit may comprise reaction vessels, each with one or more of the various reagents, preferably in concentrated form, utilized in the methods. The reagents may comprise, but are not limited to the following: low modified salt buffer, appropriate nucleotide triphosphates (e.g. dATP, dCTP, dGTP, dTTP; or rATP, rCTP, rGTP, and UTP) reverse transcriptase, RNase H, thermal stable DNA polymerase, RNA polymerase, DNA polymerase, ligase. RNase inhibitors and the appropriate primer complexes. In addition, the reaction vessels in the kit may comprise 0.2-1.0 ml tubes capable of fitting a standard thermocycler, which may be available singly, in strips of 8, 12, 24, 48, or 96 well plates depending on the quantity of reactions desired. Hence, the amplification of nucleic acids may be automated, e.g., performed in a PCR theromcycler. The thermocyclers may include, but are not limited to the following: Perkin Elmer 9600, MJ Research PTC 200, Techne Gene E, Erichrom, and Whatman Biometra T1 Thermocycler.

Also, the automated machine of the present invention may include an integrated reaction device and a robotic delivery system. In such cases, part of all of the operation steps may automatically be done in an automated cartridge. (See U.S. Pat. Nos. 5,856,174, 5,922,591, and 6,043,080.)

Without further elaboration, one skilled in the art with the preceding description can utilize the present invention to its fullest extent. The following examples are illustrative only, and not intended to limit the remainder of the disclosure in any way.

### EXAMPLES

### Example 1

### One-Cycle cDNA Synthesis Protocol Without cDNA Cleanup

Step 1: Preparation of Poly-A RNA Controls for One-Cycle cDNA Synthesis.

The GeneChip Eukaryotic Poly-A RNA Control Kit may be used for this step. The kit provides exogenous positive controls to monitor the entire GeneChip eukaryotic target labeling process, a set of GeneChip Eukaryotic Poly-A RNA Controls are supplied in the GeneChip Eukaryotic Poly-A RNA Control Kit. Each eukaryotic GeneChip probe array contains probe sets for several B. subtilis genes that are absent in eukaryotic samples (lys, phe, thr and dap). These poly-A RNA Controls are in vitro synthesized, and the polyadenylated transcripts for these B. subtilis genes are pre-mixed at staggered concentrations. The concentrated Poly-A Control Stock can be diluted with the Poly-A Control Dil Buffer and spiked directly into RNA samples to achieve the final concentrations (referred to as a ratio of copy number, for example 1:100,000 indicates that there is approximately 1 copy of the lys transcript per 100,000 transcripts in the sample) summarized below:

| | Final Concentration (ratio of |
|---|---|
| Poly-A RNA spike | copy number) |
| lys | 1:100,000 |
| phe | 1:50,000 |
| thr | 1:25,000 |
| dap | 1:7,500 |

The controls are added to the total RNA sample prior to amplification and then amplified and labeled together with the samples. Examining the hybridization intensities of these controls on GeneChip arrays may be used to monitor the labeling process independently from the quality of the starting RNA samples. The Poly-A RNA Control Stock and Poly-A Control Dil Buffer are provided with the kit to prepare the appropriate serial dilutions based on the table below. The poly-A control stocks can also be synthesized from plasmids containing the genes. These plasmids are available from the ATCC as pGIBS-lys (ATCC 87482), pGIBS-phe (ATCC 87483), pGIBS-thr (ATCC 87484), and pGIBS-dap (ATCC 87486). This is a guideline when 1, 5 or 10 μg of total RNA or 0.2 μg of mRNA is used as starting material. For other starting sample amounts, calculations are needed in order to perform the appropriate dilutions to arrive at the same proportionate final concentration of the spike-in controls in the samples. Non-stick RNase-free microfuge tubes may be used to prepare dilutions. Avoid pipetting solutions less than 2 μL in volume to maintain precision and consistency when preparing the dilutions.

Starting Amount

| | | |
|---|---|---|
| Total | Serial dilutions | Spike- |

| RNA | mRNA | First | Second | Third | in volume |
|---|---|---|---|---|---|
| 1 μg | | 1:20 | 1:50 | 1:50 | 2 μL |
| 5 μg | | 1:20 | 1:50 | 1:10 | 2 μL |
| 10 μg | 0.2 μg | 1:20 | 1:50 | 1:5 | 2 μL |

For example, to prepare the poly-A RNA dilution for 5 μg of total RNA: Add 2μL of the Poly-A Control Stock to 38 μL of Poly-A Control Dil Buffer for the First Dilution (1:20). Mix thoroughly and spin down to collect the liquid at the bottom of the tube. Add 2 μL of the First Dilution to 98 μL of Poly-A Control Dil Buffer to prepare the Second Dilution (1:50). Mix thoroughly and spin down to collect the liquid at the bottom of the tube. Add 2 μL of the Second Dilution to 18 μL of Poly-A Control Dil Buffer to prepare the Third Dilution (1:10). Mix thoroughly and spin down to collect the liquid at the bottom of the tube. Add 2 μL of this Third Dilution to 5 μg of sample total RNA.

Step 2: 1^{st} Strand cDNA Synthesis

The Affymetrix One-Cycle cDNA Synthesis Kit may be used for this step. All of the incubations may be performed in thermal cyclers. The following program can be used as a reference to perform the 1^{st} strand cDNA synthesis reaction in a thermal cycler; the 4° C. holds are for reagent addition steps: 70° C. for 10 minutes, 4° C. to hold, 42° C. for 1 hour and 4° C. to hold.

Mix the RNA sample, diluted poly-A RNA Controls, and T7-Oligo(dT) Primer. Bring the final volume to 8 μL with RNase-free Water. Incubate for 10 minutes at 70° C.; then cool the sample at 4° C. for at least 2 minutes. Place total RNA in a 0.2 mL PCR tube. Add 2 μL of the appropriately diluted poly-A RNA Controls (See Step 1). Add 2 μL of T7-Oligo(dT) Primer. Add RNase-free Water to a final volume of 8 μL. Gently flick the tube a few times to mix, and then centrifuge briefly (˜5 seconds) to collect the reaction at the bottom of the tube. Incubate the reaction for 10 minutes at 70° C. Cool the sample at 4° C. for at least 2 minutes. Centrifuge the tube briefly (˜5 seconds) to collect the sample at the bottom of the tube.

In a separate tube, assemble the 1^{st} Strand Master Mix. Prepare sufficient 1^{st} Strand Master Mix for all of the RNA samples. When there are more than 2 samples, it is prudent to include additional material to compensate for potential pipetting inaccuracy or solution lost during the process. The following recipe is for a single reaction: 3 μL 5×1^{st} Strand Reaction Mix, 1.5 μL DTT, 0.1 M, 0.75 μL dNTP, 10 mM, 1.25 μL SuperScript™ II (200U/uL), 0.5 μL RNase Inhibitor in a total volume of 7.00 μL. Mix well by flicking the tube a few times. Centrifuge briefly (˜5 seconds) to collect the master mix at the bottom of the tube. Transfer 7 μL of 1^{st} Strand Master Mix to each RNA sample. Transfer 7 μL of 1^{st} Strand Master Mix to each RNA sample/poly-A Control/T7-Oligo(dT) Primer mix for a final volume of 15 μL. Mix thoroughly by flicking the tube a few times. Centrifuge briefly (˜5 seconds) to collect the reaction at the bottom of the tube, and immediately place the tubes at 42° C. Incubate for 60 minutes at 42° C., then cool the sample for at least 2 minutes at 4° C. Cool the samples at 4° C. before proceeding to the next step. Adding the 2^{nd} Strand Master Mix directly to solutions that are at 42° C. may compromise enzyme activity. After incubation at 4° C., centrifuge the tube briefly (˜5 seconds) to collect the reaction at the bottom of the tube and immediately proceed to Step 3.

Step 3: 2^{nd} Strand cDNA Synthesis

The following program can be used as a reference to perform the 2^{nd} strand cDNA synthesis reaction in a thermal cycler: 16° C. for 2 hours, 4° C. to hold, 16° C. for 5 minutes, 75° C. for 10 minutes and hold at 4° C. In a separate tube, assemble 2^{nd} Strand Master Mix, preferably immediately before use. Prepare sufficient 2^{nd} Strand Master Mix for all of the samples. When there are more than 2 samples, it is prudent to include additional material to compensate for potential pipetting inaccuracy or solution lost during the process. The following recipe is for a single reaction: 2.75 μL RNase-free Water, 1.1 μL 50 mM MgCl₂, 0.4 μL dNTP, 10 mM, 0.6 μL E. coli DNA Polymerase I (10U/uL), 0.15 μL RNase H (2U/uL) in a Total Volume of 5.00 μL. (Prepare fresh from 1M MgCl₂ (50 μL 1M MgCl2+950 μL water)). Mix well by gently flicking the tube a few times. Centrifuge briefly (˜5 seconds) to collect the solution at the bottom of the tube. Transfer 5 μL of 2^{nd} Strand Master Mix to each sample. Add 5 μL of 2^{nd} Strand Master Mix to each 1^{st} strand synthesis sample from step 1 above for a total volume of 20 μL. Gently flick the tube a few times to mix, and then centrifuge briefly (˜5 seconds) to collect the reaction at the bottom of the tube. Incubate for 2 hours at 16° C. Add 1 μL of T4 DNA Polymerase (5U/uL) per sample and incubate for 5 minutes at 16° C. Then, 10 minutes at 75° C. Cool samples at 4° C. The reaction should not be left at 4° C. for long periods of time.

In some embodiments the double stranded template DNA is subjected to a cleanup step before being used in the next step. The cleanup may be any method known in the art, for example a column may be used or phenol extraction and ethanol precipitation. This cleanup is optional and is not included in the preferred embodiment. In the preferred embodiment a heat inactivation step is included instead of cleanup.

Synthesis of Biotin-Labeled cRNA for One-Cycle Assay

In a preferred embodiment the GeneChip IVT Labeling Kit is used for labeling cRNA. Transfer an appropriate amount of cDNA to an RNase-free microfuge tube and add the following reaction components in the order indicated at room temp. RNase-free water for a final volume of 40 μL, 4 μL 10×IVT Labeling buffer, 12 μL IVT Labeling NTP mix, and 4 μL IVT Labeling enzyme mix. Mix the reagents and collect the mixture at the bottom by brief microcentrifugation. Incubate at 37° C. for 16 hours. In a preferred embodiment, incubations are performed in an oven incubator or in a thermal cycler to reduce condensation. Labeled cRNA may be stored at −20° C. or −70° C. if not purified immediately.

In another embodiment the Enzo® BioArray™ HighYield™ RNA Transcript Labeling Kit may be used for this step to generate labeled cRNA target. The purity and quality of template cDNA is important for high yields of biotin-labeled RNA. Use only RNase-free Water, buffers, and pipette tips. Store all reagents in a −20° C. freezer that is not self-defrosting. Prior to use, centrifuge all reagents briefly to ensure that the components remain at the bottom of the tube. The product should be used only until the expiration date stated on the label. Add to cDNA from Step 3 substep 4 above 18 uL of the following master mix: 22 μL Template cDNA, 4 μL 10×HY Reaction Buffer (Vial 1), 4 μL 10×Biotin-Labeled Ribonucleotides (Vial 2), 4 μL 10×DTT (Vial 3), 4 μL 10×RNase Inhibitor Mix (Vial 4), and 2 μL 20×T7 RNA Polymerase (Vial 5) for a total volume of 40 μL. Carefully mix the reagents and collect the mixture in the bottom of the tube by brief (5 second) microcentrifugation. Immediately incubate the tube at 37° C. in a thermal cycler. For incubation time use the following times: starting material of 1.0 μg total RNA incubate 16 hours for 5 to 15 μg total RNA starting material incubate 4 hours. Store labeled cRNA at −20° C. or −70° C. if not purifying immediately, or proceed to Cleanup and Quantification of Biotin-Labeled cRNA.

Cleanup and Quantification of Biotin-Labeled cRNA for One-Cycle Assay is done using the GeneChip® Sample Cleanup Module. Reagents to be supplied by the user are Ethanol, 96-100% (v/v) and Ethanol, 80% (v/v), all other components needed for cleanup of biotin-labeled cRNA are supplied with the GeneChip Sample Cleanup Module.

Step 1: Cleanup of Biotin-Labeled cRNA

In some embodiments, removal of unincorporated NTPs is performed so that the concentration and purity of cRNA can be accurately determined by 260 nm absorbance. Biotin-labeled RNA is preferably not extracted with phenol-chloroform, because the biotin may cause some of the RNA to partition into the organic phase, lowering yields. If possible, save an aliquot of the unpurified IVT product for analysis by gel electrophoresis.

The IVT cRNA Wash Buffer is supplied as a concentrate. Before using for the first time, 20 mL of ethanol is added (96-100%), as indicated on the bottle, to obtain a working solution. The bottle may be labeled accordingly to avoid confusion.

IVT cRNA Binding Buffer may form a precipitate upon storage. If necessary, redissolve by warming in a water bath at 30° C., and then place the buffer at room temperature. All steps of the protocol are preferably performed at room temperature. It is preferable if during the procedure, the researcher can work without interruption.

The steps are as follows: 1. Add 60 μL of RNase-free Water to the IVT reaction and mix by vortexing for 3 seconds. 2. Add 350 μL IVT cRNA Binding Buffer to the sample and mix by vortexing for 3 seconds. 3. Add 250 μL ethanol (96-100%) to the lysate, and mix well by pipetting. Do not centrifuge. 4. Apply sample (700 μL) to the IVT cRNA Cleanup Spin Column sitting in a 2 mL Collection Tube. Centrifuge for 15 seconds at ≧8,000×g (≧10,000 rpm). Discard flow-through and Collection Tube. 5. Transfer the spin column into a new 2 mL Collection Tube (supplied). Pipet 500 μL IVT cRNA Wash Buffer onto the spin column. Centrifuge for 15 seconds at ≧8,000×g (≧10,000 rpm) to wash. Discard flow-through. [IVT cRNA Wash Buffer is supplied as a concentrate. Ensure that ethanol is added to the IVT cRNA Wash Buffer before use (see IMPORTANT note above before starting).] 6. Pipet 500 μL 80% (v/v) ethanol onto the spin column and centrifuge for 15 seconds at ≧8,000×g (≧10,000 rpm). Discard flow-through. 7. Open the cap of the spin column and centrifuge for 5 minutes at maximum speed (≦25,000×g). Discard flow-through and Collection Tube. Columns may be placed into the centrifuge using every second bucket. Position caps over the adjoining bucket so that they are oriented in the opposite direction to the rotation (i.e., if the microcentrifuge rotates in a clockwise direction, orient the caps in a counterclockwise direction). This avoids damage of the caps. The collection tube may be labeled with the sample name. During centrifugation some column caps may break, resulting in loss of sample information. Centrifugation with open caps allows complete drying of the membrane. 8. Transfer spin column into a new 1.5 mL Collection Tube, and pipet 11 μL of RNase-free Water directly onto the spin column membrane. Ensure that the water is dispensed directly onto the membrane. Centrifuge 1 minute at maximum speed (≦25,000×g) to elute. 9. Pipet 10 μL of RNase-free Water directly onto the spin column membrane. Ensure that the water is dispensed directly onto the membrane. Centrifuge 1 minute at maximum speed (≦25,000×g) to elute. For subsequent photometric quantification of the purified cRNA the eluate may be diluted to between 1:100 fold and 1:200 fold.

Step 2: Quantification of the cRNA

Spectrophotometric analysis may be used to determine the cRNA yield. Apply the convention that 1 absorbance unit at 260 nm equals 40 μg/mL RNA._Check the absorbance at 260 nm and 280 nm to determine sample concentration and purity. Maintain the A₂₆₀/A₂₈₀ ratio close to 2.0 for pure RNA (ratios between 1.9 and 2.1 are acceptable). The amount of cRNA required for one array hybridization experiment varies depending on the array format. Please refer to a specific probe array package insert for information on the array format.

In preferred embodiments the minimum concentration for purified cRNA is 0.6 μg/μL before starting the following fragmentation reaction in “Fragmenting the cRNA for Target Preparation” described in the GeneChip Expression Analysis Technical Manual.

### Example 2

### Two-Cycle cDNA Synthesis Without cDNA Cleanup

Step 1: Preparation of Poly-A RNA Controls for Two-Cycle cDNA Synthesis

GeneChip Eukaryotic Poly-A RNA Control Kit is used for this step. This kit is designed specifically to provide exogenous positive controls to monitor the entire GeneChip eukaryotic target labeling process, a set of GeneChip Eukaryotic Poly-A RNA Controls are supplied as GeneChip Eukaryotic Poly-A RNA Control Kit, available from Affymetrix, Santa Clara. Each eukaryotic GeneChip probe array contains probe sets for several B. subtilis genes that are absent in eukaryotic samples (lys, phe, dap and thr). These Poly-A RNA Controls are in vitro synthesized, and the polyadenylated transcripts for these B. subtilis genes are pre-mixed at staggered concentrations. The concentrated Poly-A Control Stock can be diluted with the Poly-A Control Dil Buffer and spiked directly into the RNA samples to achieve the final concentrations (referred to as a ratio of copy number) summarized below:

| | Final Concentration |
|---|---|
| Poly-A RNA | (ratio of copy |
| spike | number) |
| lys | 1:100,000 |
| phe | 1:50,000 |
| thr | 1:25,000 |
| dap | 1:7,500 |

The controls are then amplified and labeled together with the samples. Examining the hybridization intensities of these controls on GeneChip arrays helps to monitor the labeling process independently from the quality of the starting RNA samples.

The Poly-A RNA Control Stock and Poly-A Control Dil Buffer are provided with the kit to prepare the appropriate serial dilutions based on the table below. This is a guideline when 10, 50 or 100 ng of total RNA is used as starting material. For other intermediate starting sample amounts, calculate the appropriate dilutions to arrive at the same proportionate final concentration of the spike-in controls in the samples. Non-stick RNase-free microfuge tubes are preferably used to prepare the dilutions.

| | | | | | Volume |
|---|---|---|---|---|---|
| | | | | | into 50 |
| | | | | | μM T7- |
| Starting | Serial dilutions | | | | Oligo(dT) |
| ng of total RNA | First | Second | Third | Fourth | Promoter Primer |
| 10 ng | 1:20 | 1:50 | 1:50 | 1:10 | 2 μL |
| 50 ng | 1:20 | 1:50 | 1:50 | 1:2 | 2 μL |
| 100 ng | 1:20 | 1:50 | 1:50 | | 2 μL |

For example, to prepare the poly-A RNA dilution for 10 ng of total RNA: Add 2μL of the Poly-A Control Stock to 38 μL of Poly-A Control Dil Buffer to prepare the First Dilution (1:20). Mix thoroughly and spin down to collect the liquid at the bottom of the tube. Add 2 μL of the First Dilution to 98 μL of Poly-A Control Dil Buffer to prepare the Second Dilution (1:50). Mix thoroughly and spin down to collect the liquid at the bottom of the tube. Add 2 μL of the Second Dilution to 98 μL of Poly-A Control Dil Buffer to prepare the Third Dilution (1:50). Mix thoroughly and spin down to collect the liquid at the bottom of the tube. Add 2 μL of the Third Dilution to 18 μL of Poly-A Control Dil Buffer to prepare the Fourth Dilution (1:10). Use the Fourth Dilution to prepare the solution described below.

Small Sample vII requires a fresh dilution of the T7-Oligo(dT) Promoter Primer from 50 μM to 5 μM. The diluted Poly-A RNA controls should be added to the concentrated Promoter Primer as follows, using a non-stick RNase-free microfuge tube: 2 μL T7-Oligo(dT) Promoter Primer, 2 μL Diluted Poly-A RNA Controls and 16 μL Nuclease-free Water. The first dilution of the poly-A RNA controls (1:20) can be stored in a non frost-free freezer at −20° C. for at least 1.5 months and frozen-thawed at least 8 times.

Step 2: 1^{st} Cycle, 1^{st} Strand cDNA Synthesis

The following program can be used as a reference to perform the 1^{st} Cycle, 1^{st} Strand cDNA synthesis reaction in a thermal cycler; the 4° C. holds are for reagent addition steps: 6 minutes at 70° C., hold at 4° C., 1 hour at 42° C., 10 minutes at 70° C. and hold at 4° C.

Mix sample RNA and T7-Oligo(dT) Promoter Primer-Poly-A control mix. Bring the final volume to 5 μL with Nuclease-free Water. Incubate for 6 minutes at 70° C.; then cool the sample for at least 2 minutes at 4° C. Mix the total RNA sample, which is in a variable volume (10-100 ng) with 2 μL T7-Oligo(dT) Promoter Primer-Poly-A control mix and bring the final volume to 5 μL with Nuclease-free Water as follows: place total RNA samples (10 to 100 ng) in a 0.2 mL PCR tube, add 2 μL of the T7-Oligo(dT) Promoter Primer-Poly-A Control mix [See Step 1: Preparation of Poly-A RNA Controls for Two-Cycle cDNA Synthesis], add Nuclease-free Water to a final volume of 5 μL. Gently flick the tube a few times to mix, then centrifuge the tubes briefly (˜5 seconds) to collect the solution at the bottom of the tube. Incubate for 6 minutes at 70° C. Cool the sample at 4° C. for at least 2 minutes. Centrifuge briefly (˜5 seconds) to collect the sample at the bottom of the tube.

In a separate tube, assemble the 1^{st} Cycle, 1^{st} Strand Master Mix at room temperature. Prepare sufficient 1^{st} Cycle, 1^{st} Strand Master Mix for all of the total RNA samples. When there are more than 2 samples, it is prudent to include additional material to compensate for potential pipetting inaccuracy or solution lost during the process. The following recipe is for a single reaction: 2.0 μL 5×1^{st} Strand buffer, 1.0 μL 0.1 M DTT, 0.5 μL RNase OUT, 0.5 μL 10 mM dNTP mix, 1.0 SuperScript II RNase H minus RT for a total volume of 5.0 μL. Mix well by gently flicking the tube a few times. Centrifuge briefly (˜5 seconds) to collect the solution at the bottom of the tube.

Transfer 5 μL of 1^{st} Cycle, 1^{st} Strand Master Mix to each RNA sample/poly-A RNA controls/T7 Oligo(dT) Primer mix from previous step for a final volume of 10 μL. Mix thoroughly by gently flicking the tube a few times. Centrifuge briefly (˜5 seconds) to collect the reaction at the bottom of the tube, and immediately place the tubes at 42° C. and incubate for 1 hour before proceeding to the next step.

Heat the sample at 70° C. for 10 minutes to inactivate the RT enzyme; then cool the sample for at least 2 minutes at 4° C. After the 2 minute incubation at 4° C., centrifuge the tube briefly (˜5 seconds) to collect the reaction at the bottom of the tube and immediately proceed to Step 3: 1^{st} Cycle, 2^{nd} strand cDNA synthesis below. Cooling the sample at 4° C. is preferable before proceeding to the next step. Adding the 1^{st} Cycle, 2^{nd} Strand Master Mix directly to solutions that are at 70° C. may compromise enzyme activity.

Step 3: 1^{st} Cycle, 2^{nd} Strand cDNA Synthesis

The following program can be used as a reference to perform the 1^{st} Cycle, 2^{nd} strand cDNA synthesis reaction in a thermal cycler. For the 16° C. incubation, turn the heated lid function off. If the heated lid function cannot be turned off, leave the lid open. Use the heated lid for the 75° C. incubation. The program is as follows: 2 hours at 16° C., 10 minutes at 75° C. and hold at 4° C. In a separate tube, assemble the 1^{st} Cycle, 2^{nd} Strand Master Mix at room temperature. It is recommended to prepare this 1^{st} Cycle, 2^{nd} Strand Master Mix immediately before use. Prepare sufficient 1^{st} Cycle, 2^{nd} Strand Master Mix for all of the total RNA samples. When there are more than 2 samples, it is prudent to include additional material to compensate for potential pipetting inaccuracy or solution lost during the process. The following recipe is for a single reaction: 4.8 μL Nuclease-free Water, 4.0 μL 17.5 mM MgCl₂, 0.4 μL dNTP mix, 0.6 μL DNA Polymerase 10U/μL, and 0.2 μL RNase H 2U/μL for a total volume of 10 μL. A fresh dilution of the MgCl₂ may be made each time by mixing 2 μL of 1M MgCl₂ with 112 μL of nuclease-free water.

Mix the master mix well by gently flicking the tube a few times. Centrifuge briefly (˜5 seconds) to collect the solution at the bottom of the tube.

At room temperature, add 10 μL of the 1^{st} Cycle, 2^{nd} Strand Master Mix to each sample from Step 2: 1^{st} Cycle, 1^{st} Strand cDNA Synthesis reaction for a total volume of 20 μL. Gently flick the tube a few times to mix, and then centrifuge briefly (˜5 seconds) to collect the reaction at the bottom of the tube. Incubate for 2 hours at 16° C., then 10 minutes at 75° C. and cool the sample at least 2 minutes at 4° C. After the 2 minute incubation at 4° C., centrifuge the tube briefly (˜5 seconds) to collect the reaction at the bottom of the tube. Proceed to Step 4: 1^{st} Cycle, IVT Amplification of cRNA or store at −20° C. In this embodiment the double stranded cDNA is not subjected to a cleanup step prior to IVT amplification, instead the enzymes are heat inactivated, but in other embodiments it may be desirable to include at least a partial purification step at this point. Purification may be by any method known in the art, for example, a column or phenol extraction with ethanol precipitation.

Step 4: 1^{st} Cycle, IVT Amplification of cRNA using MEGAScript Kit

The following program can be used as a reference to perform the 1^{st} Cycle, IVT Amplification of cRNA reaction in a thermal cycler: 16 hours at 37° C. and hold at 4° C. In a separate tube, assemble the 1^{st} Cycle, IVT Master Mix at room temperature. Prepare sufficient 1^{st} Cycle, IVTMaster Mix for all of the samples. When there are more than 2 samples, it is prudent to include additional material to compensate for potential pipetting inaccuracy or solution lost during the process. The following recipe is for a single reaction: 5 μL 10×Reaction Buffer, 5 μL T7 ATP solution, 5 μL T7 CTP solution, 5 μL T7 UTP solution, 5 μL T7 GTP solution, and 5 μL 10×Enzyme Mix for a total volume of 30 μL. Mix well by gently flicking the tube a few times. Centrifuge briefly (˜5 seconds) to collect the solution at the bottom of the tube.

At room temperature, add 30 μL of the 1^{st} Cycle, IVT Master Mix to each 20 μL of cDNA sample from Step 3 for a final volume of 50 μL. Gently flick the tube a few times to mix, then centrifuge briefly (˜5 seconds) to collect the reaction at the bottom of the tube. Incubate for 16 hours at 37° C. After the 16 hr incubation at 37° C., centrifuge the tube briefly (˜5 seconds) to collect the reaction at the bottom of the tube. The sample is now ready to be purified in Step 5: 1^{st} Cycle, Cleanup of cRNA. Alternatively, samples may be stored at −20° C. for later use.

Step 5: 1^{st} Cycle, Cleanup of cRNA using GeneChip® Sample Cleanup Module.

User supplies Ethanol, 96-100% (v/v) and Ethanol, 80% (v/v). All other components needed for cleanup of cRNA are supplied with the GeneChip Sample Cleanup Module. IVT cRNA Wash Buffer is supplied as a concentrate. Before using for the first time, add 20 mL of ethanol (96-100%), as indicated on the bottle, to obtain a working solution, and checkmark the box on the left-hand side of the bottle label to avoid confusion. IVT cRNA Binding Buffer may form a precipitate upon storage. If necessary, redissolve by warming in a water bath at 30° C., and then place the buffer at room temperature. All steps of the protocol should be performed at room temperature. During the procedure, work without interruption if possible.

Add 50 μL of RNase-free water to the IVT reaction and mix by vortexing for 3 seconds. Add 350 μL IVT cRNA Binding Buffer to the sample and mix by vortexing for 3 seconds. Add 250 μL ethanol (96-100%) to the lysate, and mix well by pipetting. Do not centrifuge. Apply sample (700 μL) to the IVT cRNA Cleanup Spin Column sitting in a 2 mL Collection Tube. Centrifuge for 15 seconds at ≧8,000×g (≧10,000 rpm). Discard flow-through and Collection Tube. Transfer the spin column into a new 2 mL Collection Tube (supplied). Pipet 500 μL IVT cRNA Wash Buffer onto the spin column. Centrifuge for 15 seconds at ≧8,000×g (≧10,000 rpm) to wash. Discard flow-through. Pipet 500 μL 80% (v/v) ethanol onto the spin column and centrifuge for 15 seconds at ≧8,000×g (≧10,000 rpm). Discard flow-through. Open the cap of the spin column and centrifuge for 5 minutes at maximum speed (≦25,000×g). Discard flow-through and Collection Tube. Place columns into the centrifuge using every second bucket. Position caps over the adjoining bucket so that they are oriented in the opposite direction to the rotation (i.e. If the microcentrifuge rotates in a clockwise direction, orient the caps in a counterclockwise direction). This avoids damage of the caps. The collection tubes are preferably labeled with the sample name. During centrifugation some column caps may break, resulting in loss of sample information. Centrifugation with open caps allows complete drying of the membrane. Transfer spin column into a new 1.5 mL Collection Tube (supplied), and pipet 13 μL of RNase-free Water directly onto the spin column membrane. Ensure that the water is dispensed directly onto the membrane. Centrifuge 1 minute at maximum speed (≦25,000×g) to elute. The average volume of eluate is 11 μL from 13 μL RNase-free Water.

To determine cRNA yield for samples starting with 50 ng or higher, remove 2 μL of the cRNA, and add 78 μL of water to measure the absorbance at 260 nm. Use 600 ng of cRNA in the second cycle. For starting material less than 50 ng, or if the yield is less than 600 ng use up to 6.5 μL of eluate for the second cycle of cDNA synthesis. Samples can be stored at −20° C. for later use, or proceed to Step 6: 2^{nd} Cycle, 1^{st} Strand cDNA Synthesis described below.

Step 6: 2^{nd} Cycle, 1^{st} Strand cDNA Synthesis

The following program can be used as a reference to perform the 2^{nd} Cycle, 1^{st} Strand cDNA synthesis reaction in a thermal cycler; the 4° C. holds are for reagent addition steps: 70° C. for 10 minutes, hold at 4° C., 42° C. for 1 hour, hold at 4° C., 37° C. for 20 minutes, 95° C. for 5 minutes and hold at 4° C.

Make a fresh dilution of the random primers (final concentration 0.2 μg/μL). Mix 2 μL of random primers 3 μg/μL with 28 μL nuclease-free water. Add 1.5 μL of Random Primers to up to 6.5 μL of purified cRNA from Step 5 and add nuclease-free water for a final volume of 8 μL. Incubate for 10 minutes at 70° C. Cool the sample at 4° C. for at least 2 minutes. Centrifuge briefly (˜5 seconds) to collect the sample at the bottom of the tube.

In a separate tube, assemble the 2^{nd} Cycle, 1^{st} Strand Master Mix at room temperature as follows. Prepare sufficient 2^{nd} Cycle, 1^{st} Strand Master Mix for all of the samples. When there are more than 2 samples, it is prudent to include additional material to compensate for potential pipetting inaccuracy or solution lost during the process. The following recipe is for a single reaction: 3 μL 5×1^{st} Strand buffer, 1.5 μL DTT 0.1 M, 0.75 μL RNase OUT, 0.75 μL dNTP mix, 1 μL SuperScript II for a total volume of 7 μL. Mix well by gently flicking the tube a few times. Centrifuge briefly (˜5 seconds) to collect the solution at the bottom of the tube.

Transfer 7 μL of 2^{nd} Cycle, 1^{st} Strand Master Mix to each cRNA/Random Primer sample from Step 6.1 for a final volume of 15 μL. Mix thoroughly by gently flicking the tube a few times. Centrifuge briefly (˜5 seconds) to collect the reaction at the bottom of the tube and place the tubes at 42° C. immediately. Incubate for 1 hour at 42° C., and then cool the sample for at least 2 minutes at 4° C. After the incubation at 4° C., centrifuge briefly (˜5 seconds) to collect the reaction at the bottom of the tube. Add 1 μL of RNase H 2U/μL to each sample for a final volume of 21 μL. Mix thoroughly by gently flicking the tube a few times. Centrifuge briefly (˜5 seconds) to collect the reaction at the bottom of the tube and incubate for 20 minutes at 37° C. Heat the sample at 95° C. for 5 minutes. Cool the sample for at least 2 minutes at 4° C.; then, proceed directly to Step 7: 2^{nd} Cycle, 2^{nd} Strand cDNA Synthesis described below.

Step 7. 2^{nd} Cycle, 2^{nd} Strand cDNA Synthesis

The following program can be used as a reference to perform the 2^{nd} Cycle, 2^{nd} Strand cDNA Synthesis reaction in a thermal cycler: 70° C. for 6 minutes, hold at 4° C., 16° C. for 2 hours, hold at 4° C., 16° C. for 10 minutes, 75° C. for 10 minutes and hold at 4° C. r the 16° C. incubation, turn the heated lid function off. If the heated lid function cannot be turned off, leave the lid open. Use the heated lid for the 75° C. incubation. The 4° C. holds are for reagent addition steps.

Make a fresh dilution of the T7-Oligo (dT) Promoter Primer (final concentration 5 μM). Mix 2 μL of T7-Oligo (dT) primer 50 μM with 18 μL of nuclease-free water. Add 2 μL of diluted T7-Oligo (dT) Promoter Primer to the sample from Step 6 for a final volume of 25 μL. Gently flick the tube a few times to mix, and then centrifuge briefly (˜5 seconds) to collect the reaction at the bottom of the tube. Incubate for 6 minutes at 70° C. Cool the sample at 4° C. for 2 minutes. Centrifuge briefly (˜5 seconds) to collect sample at the bottom of the tube. Cooling the samples at 4° C. is done before proceeding to the next step. Adding the 2^{nd} Strand Master Mix directly to solutions that are at 70° C. may compromise enzyme activity.

It is recommended to prepare the 2^{nd} Cycle, 2^{nd} Strand Master Mix immediately before use. In a separate tube, assemble the 2^{nd} Cycle, 2^{nd} Strand Master Mix at room temperature. Prepare sufficient 2^{nd} Cycle, 2^{nd} Strand Master Mix for all of the samples. When there are more than 2 samples, it is prudent to include additional material to compensate for potential pipetting inaccuracy or solution lost during the process. The following recipe is for a single reaction: 1 μL 55 mM MgCl₂, 0.4 μL dNTP 10 mM, and 0.6 μL DNA polymerase 10U/μL for a total volume of 2.0 μL. A fresh dilution of MgCl2 may be prepared by mixing 4 μL of 1M MgCl2 and 69 μL of water. Mix well by gently flicking the tube a few times. Centrifuge briefly (˜5 seconds) to collect the master mix at the bottom of the tube.

At room temperature, add 2 μL of the 2^{nd} Cycle, 2^{nd} Strand Master Mix to each sample from Step 7: 2^{nd} Cycle, 1^{st} Strand cDNA Synthesis reaction for a total volume of 20 μL. Gently flick the tube a few times to mix, then centrifuge briefly (˜5 seconds) to collect the reaction at the bottom of tube. Incubate for 2 hours at 16° C., and then cool the sample for at least 2 minutes at 4° C.

Add 2 μL of T4 DNA polymerase 5U/μL to the samples for a final volume of 22 μL. Gently flick the tube a few times to mix, and then centrifuge briefly (˜5 seconds) to collect the reaction at the bottom of the tube. Incubate for 10 minutes at 16° C. Then, 10 minutes at 75° C. Cool the sample at 4° C. for at least 2 minutes. Centrifuge briefly (˜5 seconds) to collect sample at the bottom of the tube.

In some embodiments the double stranded template DNA is subjected to a cleanup step before being used in the next step. The cleanup may be any method known in the art, for example a column may be used or phenol extraction and ethanol precipitation. This cleanup is optional and is not included in the preferred embodiment. In the preferred embodiment a heat inactivation step is included instead of cleanup.

Step 8. Synthesis of Biotin-Labeled cRNA

In a preferred embodiment the GeneChip IVT Labeling Kit is used for labeling cRNA. Transfer an appropriate amount of cDNA to an RNase-free microfuge tube and add the following reaction components in the order indicated at room temp. RNase-free water for a final volume of 40 μL, 4 μL 10×IVT Labeling buffer, 12 μL IVT Labeling NTP mix, and 4 μL IVT Labeling enzyme mix. Mix the reagents and collect the mixture at the bottom by brief micro centrifugation. Incubate at 37° C. for 16 hours. In a preferred embodiment, incubations are performed in an oven incubator or in a thermal cycler to reduce condensation. Labeled cRNA may be stored at −20° C. or −70° C. if not purified immediately.

The Enzo® BioArray™ HighYield™ RNA Transcript Labeling Kit may be used for this step to generate labeled cRNA target. The purity and quality of template cDNA is important for high yields of biotin-labeled RNA. Use nuclease-free water, buffers, and pipette tips. Store all reagents in a −20° C. freezer that is not self-defrosting. Prior to use, centrifuge all reagents briefly to ensure that the components remain at the bottom of the tube. The product should be used only until the expiration date stated on the label.

Add to RNase-free microfuge tubes template cDNA and additions of other reaction components in the following order: 22 μL Template cDNA, 4 μL 10×HY Reaction Buffer (Vial 1), 4 μL 10×Biotin-Labeled Ribonucleotides (Vial 2), 4 μL 10×DTT (Vial 3), 4 μL 10×RNase Inhibitor Mix (Vial 4), and 2 μL 20×T7 RNA Polymerase (Vial 5) for a total volume of 40 μL. Keep reactions at room temperature while additions are made to avoid precipitation of DTT.

Carefully mix the reagents and collect the mixture in the bottom of the tube by brief (5 second) micro centrifugation. Immediately incubate the tube at 37° C. for 4 hours in a thermal cycler. Store labeled cRNA at −20° C. or −70° C. if not purifying immediately, or proceed to Step 10: Cleanup and Quantification of Biotin-Labeled cRNA.

Step 9. Cleanup and Quantification of Biotin-Labeled cRNA

GeneChip® Sample Cleanup Module may be used for cleaning up the Biotin Labeled cRNA. Reagents to be supplied by the user are Ethanol, 96-100% (v/v) and Ethanol, 80% (v/v). All other components needed for cleanup of biotin-labeled cRNA are supplied with the GeneChip Sample Cleanup Module.

Step 9.1: Cleanup of Biotin-Labeled cRNA

Unincorporated NTPs are removed, so that the concentration and purity of cRNA can be accurately determined by 260 nm absorbance. Biotin-labeled RNA is preferably not extracted with phenol-chloroform, because the biotin may cause some of the RNA to partition into the organic phase. This will result in low yields. Save an aliquot of the unpurified IVT product for analysis by gel electrophoresis. IVT cRNA Wash Buffer is supplied as a concentrate. Before using for the first time, add 20 mL of ethanol (96-100%), as indicated on the bottle, to obtain a working solution, and checkmark the box on the left-hand side of the bottle label to avoid confusion. IVT cRNA Binding Buffer may form a precipitate upon storage. If necessary, redissolve by warming in a water bath at 30° C., and then place the buffer at room temperature. All steps of the protocol are preferably performed at room temperature. During the procedure, the user preferably works without interruption.

Add 60 μL of RNase-free water to the IVT reaction and mix by vortexing for 3 seconds. Add 350 μL IVT cRNA Binding Buffer to the sample and mix by vortexing for 3 seconds. Add 250 μL ethanol (96-100%) to the lysate and mix well by pipetting. Do not centrifuge. Apply sample (700 μL) to the IVT cRNA Cleanup Spin Column sitting in a 2 mL Collection Tube. Centrifuge for 15 seconds at ≧8,000×g (≧10,000 rpm). Discard flow-through and Collection Tube. Transfer the spin column into a new 2 mL Collection Tube (supplied). Pipet 500 μL IVT cRNA Wash Buffer onto the spin column and centrifuge for 15 seconds at ≧8,000×g (≧10,000 rpm) to wash. Discard flow-through. Pipet 500 μL 80% (v/v) ethanol onto the spin column and centrifuge for 15 seconds at ≧8,000×g (≧10,000 rpm). Discard flow-through. Open the cap of the spin column and centrifuge for 5 minutes at maximum speed (≦25,000×g). Discard flow-through and Collection Tube. Place columns into the centrifuge using every second bucket. Position caps over the adjoining bucket so that they are oriented in the opposite direction to the rotation (i.e., if the micro centrifuge rotates in a clockwise direction, orient the caps in a counterclockwise direction). This avoids damage of the caps. Label the collection tube with the sample name. During centrifugation some column caps may break, resulting in loss of sample information. Centrifugation with open caps allows complete drying of the membrane.

Transfer spin column into a new 1.5 mL Collection Tube (supplied), and pipet 11 μL of RNase-free Water directly onto the spin column membrane. Ensure that the water is dispensed directly onto the membrane. Centrifuge 1 minute at maximum speed (≦25,000×g) to elute. Pipet 10 μL of RNase-free Water directly onto the spin column membrane. Ensure that the water is dispensed directly onto the membrane. Centrifuge 1 minute at maximum speed (≦25,000×g) to elute. For subsequent photometric quantification of the purified cRNA, we recommend dilution of the eluate between 1:100 fold and 1:200 fold.

Step 9.2: Quantification of the cRNA

Use spectrophotometric analysis to determine the cRNA yield. Apply the convention that 1 absorbance unit at 260 nm equals 40 μg/mL RNA. Check the absorbance at 260 nm and 280 nm to determine sample concentration and purity. Maintain the A₂₆₀/A₂₈₀ ratio close to 2.0 for pure RNA (ratios between 1.9 and 2.1 are acceptable). The amount of cRNA required for one array hybridization experiment varies depending on the array format. Please refer to a specific probe array package insert for information on the array format. In some embodiments the minimum concentration for purified cRNA is 0.6 μg/μL before starting the following fragmentation reaction in “Fragmenting the cRNA for Target Preparation”.

The specific embodiments described above do not limit the scope of the present invention in any way as they are single illustrations of individual aspects of the invention. Functionally equivalent methods and components are within the scope of the invention. The scope of the appended claims thus includes modifications that will become apparent to those skilled in the art from the foregoing description.

1. A method for estimating the relative abundance of a plurality of mRNAs in a nucleic acid sample, said method comprising:
(a) contacting a nucleic acid sample comprising a plurality of different polyadenylated mRNAs with a first primer comprising poly d(T) and an RNA polymerase promoter; and, extending the first primer in a reaction mixture comprising reverse transcriptase to generate RNA:DNA duplexes;
(b) synthesizing second strand cDNA by incubating the RNA:cDNA duplexes with a reaction mixture comprising DNA polymerase, RNase H and dNTPs, generating double stranded cDNA comprising an RNA polymerase promoter;
(c) producing multiple copies of unlabeled antisense RNA by incubating the double stranded cDNA in a reaction mixture comprising an RNA polymerase, ATP, CTP, UTP and GTP;
(d) purifying the multiple copies of unlabeled antisense RNA;
(e) contacting the purified multiple copies of RNA with a reaction mixture comprising random primers; and, generating RNA:cDNA duplexes from the purified multiple copies of RNA by extending the random primers in a reaction mixture comprising a reverse transcriptase and dNTPs;
(f) denaturing the RNA:cDNA duplexes;
(g) contacting the DNA with a second primer comprising oligo dT and an RNA polymerase promoter and extending the second primer to generate double stranded cDNA;
(h) forming a double stranded DNA promoter region by adding the appropriate reagents;
(i) producing multiple copies of labeled antisense cRNA by an in vitro transcription reaction;
(j) fragmenting the labeled antisense cRNA;
(k) hybridizing the fragmented labeled antisense cRNA to a solid support comprising nucleic acid probes; and
(l) analyzing the hybridization pattern to provide an estimate of the relative abundance of a plurality of mRNAs in the nucleic acid sample.

2. The method of claim 1 wherein prior to step (a) a known amount of at least one polyadenylated control transcript is added to the nucleic acid sample, wherein the at least one polyadenylated control transcript is not naturally present in the nucleic acid sample.

3. The method of claim 2 wherein the at least one polyadenylated control transcript is a transcript from a gene selected from the group consisting of B. subtilis lys, phe, thr and dap and wherein the solid support further comprises probes to detect at least one transcript from B. subtilis lys, phe, thr and dap.

4. The method of claim 1 wherein prior to step (a) known amounts of a plurality of polyadenylated control transcripts are added to the nucleic acid sample, wherein each of the plurality of polyadenylated transcripts is a transcript from a gene from a prokaryotic organism.

5. The method of claim 4 wherein the prokaryotic organism is B. subtilis.

6. The method of claim 5 wherein the polyadenylated control transcripts are transcribed from the group of genes consisting of B. subtilis lys, phe, thr and dap.

7. The method of claim 4 wherein each control transcript in the plurality is added to the nucleic acid sample at a concentration that is different from the concentration of the other control transcripts in the plurality.

8. The method of claim 6 wherein the plurality of polyadenylated control transcripts consists of transcripts from B. subtilis lys, phe, thr and dap genes and wherein the control transcript from the lys gene is present at approximately 1 copy per 100,000 transcripts in the sample, the control transcript from the phe gene is present at approximately 1 copy per 50,000 transcripts in the sample, the control transcript from the thr gene is present at approximately 1 copy per 25,000 transcripts in the sample and the control transcript from the dap gene is present at approximately 1 copy per 7,500 transcripts in the sample.

9. The method of claim 1 wherein said solid support comprising nucleic acid probes is selected from the group consisting of a nucleic acid probe array, a membrane blot, a microwell, a bead, and a sample tube.

10. The method of claim 1 wherein said nucleic acid sample is obtained from blood or a buccal swab.

11. The method of claim 1 wherein the method involves the use of a thermocycler, an integrated reaction device, and a robotic delivery system.

12. A kit for the amplification of nucleic acids, wherein said kit comprises a container, instructions for use, a promoter which comprises a poly d(T) sequence operably linked to an RNA polymerase promoter and at least one polyadenylated control transcript from a gene from a prokaryotic organism.

13. The kit of claim 12 wherein the kit comprises polyadenylated control transcripts from each gene in the group of genes consisting of B. subtilis lys, phe, thr and dap genes.

14. A method for estimating the relative abundance of a plurality of mRNAs in a nucleic acid sample, said method comprising:
(a) obtaining a nucleic acid sample wherein the nucleic acid sample comprises a mixture of polyadenylated mRNAs wherein at least one of the mRNAs is present in the nucleic acid sample at unknown levels;
(b) adding a known amount of at least one polyadenylated control transcript to the nucleic acid sample, wherein the at least one polyadenylated control transcript is not naturally present in the nucleic acid sample, to form a mixed nucleic acid sample;
(c) contacting the mixed nucleic acid sample with a first primer comprising poly d(T) and an RNA polymerase promoter; and, extending the first primer in a reaction mixture comprising reverse transcriptase to generate RNA:cDNA duplexes;
(d) synthesizing second strand cDNA by incubating the RNA:cDNA duplexes with a reaction mixture comprising DNA polymerase, RNase H and dNTPs, generating double stranded cDNA comprising an RNA polymerase promoter;
(e) producing multiple copies of unlabeled antisense RNA by incubating the double stranded cDNA in a reaction mixture comprising an RNA polymerase, ATP, CTP, UTP and GTP;
(f) purifying the multiple copies of unlabeled antisense RNA;
(g) contacting the purified multiple copies of RNA with a reaction mixture comprising random primers; and, generating RNA:cDNA duplexes from the purified multiple copies of unlabeled antisense RNA by extending the random primers in a reaction mixture comprising a reverse transcriptase and dNTPs;
(h) denaturing the RNA:cDNA duplexes;
(i) contacting the cDNA with a second primer comprising oligo dT and an RNA polymerase promoter and extending the second primer to generate double stranded cDNA;
(j) forming a double stranded DNA promoter region by adding the appropriate reagents;
(k) producing multiple copies of labeled antisense cRNA by an in vitro transcription reaction;
(l) fragmenting the labeled antisense cRNA;
(m) hybridizing the fragmented labeled antisense cRNA to a solid support comprising nucleic acid probes that detect a plurality of mRNAs and nucleic acid probes that detect the at least one polyadenylated control transcript added in step (b) and
(n) analyzing the hybridization pattern of the probes that detect a plurality of mRNAs to provide an estimate of the relative abundance of a plurality of mRNAs in the nucleic acid sample and analyzing the hybridization pattern of the probes that detect the at least one control transcript to estimate the efficiency of one or more steps selected from steps (c) through (k).

15. The method of claim 14 wherein the at least one polyadenylated control transcript is selected from the group consisting of B. subtilis lys, phe, thr and dap.

16. The method of claim 14 wherein the polyadenylated control transcript is a transcript from a gene from a prokaryotic organism.

17. The method of claim 16 wherein the prokaryotic organism is B. subtilis.

18. The method of claim 16 wherein the polyadenylated transcripts are transcribed from the group of genes consisting of B. subtilis lys, phe, thr and dap.

19. The method of claim 14 wherein the polyadenylated control transcripts added in step (b) comprise transcripts from B. subtilis lys, phe, thr and dap and wherein each control transcript is added to the nucleic acid sample at a concentration that is different from the concentration of the other control transcripts added.

20. The method of claim 14 wherein a plurality of polyadenylated control transcripts are added in step (b) and the plurality of polyadenylated control transcripts consists of transcripts from B. subtilis lys, phe, thr and dap genes and wherein the control transcript from the lys gene is added at approximately 1 copy per 100,000 transcripts in the nucleic acid sample, the control transcript from the phe gene is added at approximately 1 copy per 50,000 transcripts in the nucleic acid sample, the control transcript from the thr gene is added at approximately 1 copy per 25,000 transcripts in the nucleic acid sample and the control transcript from the dap gene is added at approximately 1 copy per 7,500 transcripts in the nucleic acid sample.

21. The method of claim 14 wherein said solid support comprising nucleic acid probes is selected from the group consisting of a nucleic acid probe array, a membrane blot, a microwell, a bead, and a sample tube.

22. The method of claim 14 wherein said nucleic acid sample is obtained from tissue, blood or a buccal swab.

23. The method of claim 14 wherein the method involves the use of a thermocycler, an integrated reaction device, and a robotic delivery system.
